(19) United States
(12) Patent Application Publication
(10) Pub. No.: US 2009/0300858 A1
(43) Pub. Date: Dec. 10, 2009

(54) SEMI-PERMANENT HAIR COLOR COMPOSITION OF MATTER INCLUDING A WATERPROOF INGREDIENT

(76) Inventors: Bong-Lim Jo, Gyeonggi-do (KR); Hye-Jin Moon, Gyeonggi-do (KR)

Correspondence Address: FROMMER LAWRENCE & HAUG, 745 FIFTH AVENUE- 10TH FL., NEW YORK, NY, 10151, US

(21) Appl. No.: 12/521,809

(22) PCT Filed: Jun. 24, 2008
(86) PCT No.: PCT/KR2008/003592

§ 371 Date: Jun. 30, 2009

(30) Foreign Application Priority Data
Jun. 26, 2007 (KR) 10-2007-0062990

(51) Int. Cl. A61K 8/81

(52) U.S. Cl. 8/435

(57) ABSTRACT

Disclosed is a semi-permanent hair dyeing composition using a waterproof coating agent, in particular, a semi-permanent hair dyeing composition comprising a water-soluble dye, an acid dye, acrylate copolymer as a waterproof coating agent and N-methyl pyrrolidone as a solvent in a certain amount. The semi-permanent hair dyeing composition of the present invention has an advantage in that although the composition is not washed out after the application to the hair, the water-soluble dye fastened to the hair surface is not melted down by rainwater or sweat, and thereby, it can prevent skin and clothes from being contaminated with the dye.

### TECHNICAL FIELD

The present invention relates to a semi-permanent hair dyeing composition using a waterproof coating agent. Particularly, the present invention relates to a semi-permanent hair dyeing composition using a water-soluble dye, which is characterized by comprising an acid dye, acrylate copolymer as a waterproof coating agent and N-methyl pyrrolidone as a solvent in a certain amount. The semi-permanent hair dyeing composition of the present invention has an advantage in that although the composition is not washed out after the application to the hair, the water-soluble dye fastened to the hair surface is not dissolved by rainwater or sweat, and thereby, it can prevent skin and clothes from being contaminated with the dye.

### BACKGROUND ART

A hair dyeing composition is generally classified into three groups—temporary hair coloring, semi-permanent hair coloring and permanent hair coloring—primarily based on the degree of being fastened a dye to the hair (fastness).

A semi-permanent hair dyeing composition is also called an acid hair dye. When the dye is dissolved in an acidic aqueous solution, it is negatively charged. In case of applying such an acidic aqueous solution to the hair, amino acids of the hair surface (i.e., cuticle layer) are positively charged. Therefore, the semi-permanent hair dyeing composition is made to dye the hair by binding the negatively charged dye to the cations on the hair surface by an ionic bond.

The semi-permanent hair coloring agent is divided into an acid hair dye, a basic hair dye, a dispersion hair dye and the like according to a dye used. The most widely used hair coloring agent is an acid hair dye, and a hair dyeing composition of the present invention also uses an acid hair dye.

The acid dye is a general term for a water-soluble dye having an acid group such as sulfone, carboxyl and nitro inside the molecule, and includes azo, anthraquinone, triphenylmethane, xanthine and the like. There are more than 1,000 different kinds of acid dyes, but they are mostly azo-based dyes. The acid dye is commonly used for dyeing sheep wool and human hair and applied to the various fields of dyeing including leather, paper, celluloid, lake dye, ink, food coloring, indicator and the like. Generally, the acid dye shows better fastness than a basic dye. The acid dye can be used to dye a substance in an acid solution containing citric acid, glycolic acid or lactic acid, and the higher the acidity of a solution, i.e., the higher the hydrogen ion concentration, the better the dyeing performance.

However, since the acid hair coloring agent is in the form of not penetrating into the inside of hair (i.e., cortex) but fastening to the hair surface by an ionic bond, it has a problem that the dye is easily dissolved by rainwater or sweat, leading to skin or clothing contamination.

The semi-permanent hair coloring agent is characterized by using a dye which cannot penetrate into the inside of hair because its molecular weight and size are bigger than the gap between hair cuticles, but is negatively or positively charged in an aqueous solution. Such a property enables to bind the semi-permanent hair coloring agent with an amine or a carboxyl group of keratin, a major fibrous protein constituting hair, and thereby, dye hair by fastening to the hair surface as it coats. Unlike an oxidized permanent hair dye, since the semi-permanent hair dye shows acidity of pH 3 to 5, it does not cause hair damage due to hair swelling. Further, as it dose not have odor of ammonia, it is possible to dye the hair without further hair damage even when hair is already damaged. However, these dyes in the type of ionic bond have excellent solubility in water or other water-soluble solvents. Therefore, if the dye is not completely rinsed off after its application to hair, or the dyed hair is placed in sweat for a long time, or the dyed hair is exposed to rain, the dye fastened to the hair surface becomes dissolved, resulting in contaminating skin and clothes.

Generally, hydrophobic polymers are easy to form a strong waterproof coating. But, as can be presumed in its name, they are not compatible with an aqueous solution containing water, and thus form an opaque, sticky membrane, and not suitable for a composition comprising a water-soluble dye. Further, the acid dye has been most widely used in a semi-permanent hair dyeing agent due to its excellent fastness and variety in its colors. However, since the acid dye contains anions such as sulfone, carboxyl and nitro inside the molecule, these anions bind with cations of the hydrophobic polymer, and finally, the acid dye is precipitated in a water-soluble composition, which results in the decrease in a dyeing effect. Therefore, it has been known that the hydrophobic polymer is not suitable for a semi-permanent hair dyeing agent using an acid dye.

The present inventors have therefore endeavored to overcome the above problems of the prior art acid semi-permanent hair dyeing composition, and found the fact that if acrylate copolymer is used as a hydrophobic polymer for forming a waterproof coating in the acid semi-permanent hair dyeing composition, and simultaneously, N-methyl pyrrolidone is used as a solvent for acrylate copolymer, the dye fastened to the hair surface is surrounded with a hydrophobic waterproof coating, which results in preventing the dye from being dissolved by rainwater or sweat.

### DISCLOSURE

### [Technical Problem]

Accordingly, it is an object of the present invention to provide a new concept of a semi-permanent hair dyeing composition which can overcome the problems of compatibility between a hydrophobic polymer and water, does not induce any precipitation when an aqueous solution containing the hydrophobic polymer is added to an acid dye, exhibits excellent dyeing effect, and is not influenced by rainwater or sweat through the formation of a strong waterproof coating.

### DESCRIPTION OF THE DRAWINGS

The above and other objects, features and other advantages of embodiments of the present invention will be more clearly understood from the following detailed description taken in conjunction with the accompanying drawings, in which:

FIG. 1 is a diagram illustrating the procedure of preparing a semi-permanent hair dyeing composition in a liquid type;

FIG. 2 is a diagram illustrating the procedure of preparing a semi-permanent hair dyeing composition in a cream type;

FIG. 3 is a photograph of the hair left alone for 24 hours (FIG. 3a) and a photograph of a glass plate (FIG. 3b) according to whether a waterproof coating forming agent is used or not; and

FIG. 4 is a photograph of the hair soaked in water (FIG. 4a), a photograph of the hair soaked in an artificial sweat solution (FIG. 4b), and a photograph of a glass plate after washed with water (FIG. 4c) according to the use/non-use of a waterproof coating forming agent.

### BEST MODE

The semi-permanent hair dyeing composition using an acid dye in accordance with the present invention is characterized by comprising acrylate copolymer as a waterproof coating agent and N-methyl pyrrolidone as a solvent thereof. Hereinafter, the present invention will be explained in more detail.

The present invention relates to a new concept of a semi-permanent hair dyeing composition using an acid dye which further comprises a hydrophobic polymer capable of forming a strong waterproof coating, such that the dye fastened to the positively charged amino acids of the hair in an acid solution is not dissolved by rainwater or sweat, which results in preventing skin and clothes from contaminating. Further, the semi-permanent hair dyeing composition of the present invention can overcome the problem of compatibility between a water-soluble solution containing water and a hydrophobic polymer showing strong hydrophobic property and containing anions in its structure, prevent the acid dye containing anions from being precipitated, and does not impair other functional properties of the hair dyeing composition.

It is preferred to use acrylate copolymer as a polymer to form a waterproof coating and N-methyl pyrrolidone as a solvent to overcome the problem of compatibility between an aqueous solution and an anionic dye.

As a forming agent for a hydrophobic waterproof coating, it is preferred to use amphoteric acrylic resins having the properties of both an acid and a base. In particular, it is more preferable to use methacryloyl ethyl betaine/acrylate copolymer as an amphoteric acrylic resin, which is a strong hydrophobic polymer wherein methacrylic acid is mixed with methacryloyl ethyl betaine polymer. The methacryloyl ethyl betaine/acrylate copolymer is easy to form a strong waterproof coating in alcohol containing no water and a preparation containing an organic solvent, but it forms an opaque, sticky membrane in an aqueous solution containing water due to the lack of compatibility with water. Therefore, it has been known that it is not suitable for a preparation comprising a water-soluble dye. In addition, since the acid dye most widely used in a semi-permanent hair dyeing composition due to its high fastness and various kinds of colors includes anions such as sulfone, carboxyl and nitro inside the molecule, the acid dye is precipitated in a water-soluble composition through the formation of ionic bonds between the anions of the acid dye and the cations of methacryloyl ethyl betaine polymer, which results in the decrease in dyeing effect. Therefore, it has been known that methacryloyl ethyl betaine polymer is not suitable for a semi-permanent hair dyeing agent using an acid dye.

However, in order to overcome the problem of compatibility between acrylate copolymer used as a hydrophobic waterproof coating agent and a water-soluble semi-permanent hair dyeing composition containing water, the present invention employs N-methyl pyrrolidone (CAS No.: 872-50-4) which has been used for extracting incense of lubricating oil or as a solvent for a polymer. N-methyl pyrrolidone exhibits unique solubility that it cannot be completely dissolved both in water and in an organic solvent such as alcohol, ether, chloroform and the like, but is partially miscible with the both. Further, as N-methyl pyrrolidone has a function of lowering surface tension of a polymer showing high viscosity such as methacryloyl ethyl betaine/acrylate copolymer, the present invention intends to employ such a function of N-methyl pyrrolidone.

The semi-permanent hair dyeing agent is classified into four groups depending on the kind of a dye used therein: an acid hair dye, a basic hair dye, a disperse hair dye and a HC hair dye. Among these, since the acid semi-permanent hair dyeing agent can maintain its dyed color for the most prolonged period, it has been widely used.

The acid semi-permanent hair dyeing agent used in the present invention does not show any change in a molecular weight and physical properties due to the polymerization of a dye inside the hair (i.e., cortex), unlike an oxidized permanent hair dye. On the contrary, it can maintain its molecular weight and physical properties during the dyeing and perform dyeing by forming an ionic bond with amino acids present on the hair surface. Generally, hair proteins are composed of about 19 different kinds of amino acids. The acid semi-permanent hair dyeing agent uses amphoteric electrolyte characteristics of amino acids constituting the hair, wherein the amino acid is represented by the following Formula 1.

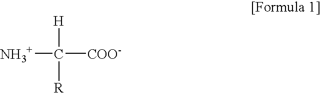

Generally, the acid hair dyeing composition has a pH of 3.0 to 4.0. When a hair dyeing agent having an acid pH is applied to the hair, carboxyl groups of amino acids are decreased due to the increase in hydrogen ion concentration within the hair dyeing composition, and thereby, amino groups are ionized much more. As a result, the amino acids of the hair get more positive charges, which makes the hair positively charged. Here, if a negatively charged dye is present in the hair dyeing composition applied to the hair, the dye can bind with the positively charged hair, and then, the hair surface is dyed. This procedure is illustrated by the following Reaction Scheme 1, with exemplifying Acid Orange 3 as an acid dye.

Generally, in the semi-permanent hair dyeing agent, the dye attached to the hair surface is washed out little by little and completely removed after four to eight times of shampoo. The acid dye is a water-soluble dye having an acid group such as oxyl (—OH), carboxyl (—COOH), sulfonic acid (—SO₃H) and the like. The dye commonly used in the acid dye is an azo-based dye. The azo-based dye has one or more azo groups (—N═N—), there are many different kinds thereof, and it has a majority of the total synthetic dyes being commercially available. The azo-based dye gives vivid and bright color to the hair and maintains the dyed color for a prolonged period. Representative examples of the azo-based dye include the following Formulae 2 to 4.

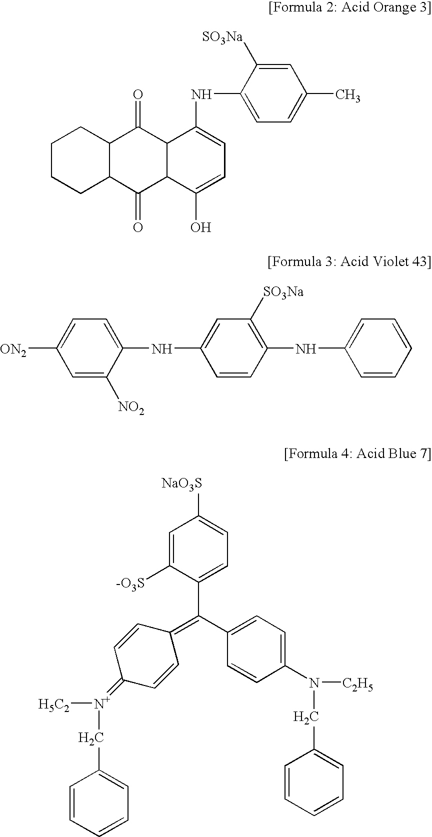

The semi-permanent hair dyeing agent using an acid dye has a pH of about 3.0 to 5.0. Since such a semi-permanent hair dyeing agent does not swell the hair and make the acid dye penetrated into a cortex layer inside the hair, allowing only a coating action to the hair surface, i.e., a cuticle layer, it has an advantage of dyeing the hair without hair damage. However, as the acid dye is dyed only to the cuticle layer, in case of being in a sweat or in the rain during daily life, there is a risk of being dissolved the highly water-soluble acid dye form the hair, and then, coloring on the skin or contaminating clothing.

The acid semi-permanent hair dyeing composition of the present invention comprises acrylate copolymer as a hydrophobic waterproof coating forming agent (waterproof coating agent) and N-methyl pyrrolidone as a solvent for the polymer, which makes possible to thoroughly mix water, a dye and a hydrophobic waterproof coating agent and prevent the dye bound to the hair surface from being dissolved by rainwater or sweat. These characteristics can be achieved by the use of N-methyl pyrrolidone for dissolving a polymer, which overcomes the problems of compatibility between water and hydrophobic acrylate copolymer and compatibility between the negatively charged dye and acrylate copolymer in an aqueous solution. Based on these facts, it has been found that acrylate copolymer forms a strong waterproof coating on the hair surface after the hair is dyed with the semi-permanent hair dyeing agent and dried.

The above waterproof coating agent can be preferably used in the amount of 1.0 to 40.0 wt % based on the total hair dyeing composition, and N-methyl pyrrolidone can be preferably used in the amount of 0.2 to 20.0 wt % based on a total hair dyeing composition. If the amount of a waterproof coating agent exceeds the above range, the viscosity of the hair dyeing composition becomes too high, which makes difficult to evenly spread it on the hair. If the amount of N-methyl pyrrolidone exceeds the above range, an oil phase and an aqueous phase of the hair dyeing composition are separated from each other.

The semi-permanent hair dyeing composition according to the present invention may further comprise additional ingredients conventionally used in an acid semi-permanent hair dyeing agent, and there is no limitation on the type of a semi-permanent hair dyeing composition regardless of whether it is in a type of liquid or cream.

The ingredients applicable to the semi-permanent hair dyeing composition of the present invention are exemplified by the following: in a liquid type, 0.1-10.0 wt % of an acid dye, 1.0-50.0 wt % of lower alcohol, 0.1-10.0 wt % of aromatic alcohol, 5.0-40.0 wt % of distilled water, 0.2-20.0 wt % of a polymer solvent, 1.0-40.0 wt % of a hydrophobic waterproof coating forming agent and 0.5-20.0 wt % of a pH regulator can be used.

Meanwhile, in a cream type, 0.1-10.0 wt % of an acid dye, 1.0-20.0 wt % of a surfactant, 2.0-25.0 wt % of aliphatic alcohol, 0.1-10.0 wt % of aromatic alcohol, 0.2-20.0 wt % of a polymer solvent, 1.0-40.0 wt % of a hydrophobic waterproof coating forming agent and 0.5-20.0 wt % of a pH regulator can be used.

As an acid dye, a water-soluble dye having an acid functional group such as sulfone, carboxyl and nitro inside the molecule can be used. Preferably, the acid dye can be at least one dye selected from the group consisting of violet 401 (Acid Violet 43, CAS No. 4430-18-6), black 401 (Acid Black 1, CAS No. 1064-48-8), orange 205 (Acid Orange 7, CAS No. 633-96-5), yellow 406 (KI 406, CAS No. 587-98-4), red 227 (CI 17200, 3567-66-6), and yellow 4 (Yellow 5, CAS No. 1934-21-0).

The lower alcohol used in the present invention is alcohol having a carbon number of 2 to 3, and preferably include ethyl alcohol, iso propyl alcohol, and the like.

The aliphatic alcohol suitable for the present invention is an aliphatic alcohol having a carbon number of 14 to 22, and can be one or more substances selected from the group consisting of myristil alcohol, cetyl alcohol, stearyl alcohol, oleyl alcohol and behenyl alcohol.

The surfactant suitable for the present invention is a non-ionic surfactant which is conventionally used in a hair dyeing agent, and representative examples thereof may include polyoxyethylene cetyl ether, polyoxyethylene cetostearyl ether and the like.

The aromatic alcohol disclosed above is preferably benzyl alcohol.

The pH regulator used in the present invention may include glycolic acid, lactic acid, citric acid and the like.

The semi-permanent hair dyeing composition prepared by using the above ingredients in accordance with the present invention can overcome the problem of compatibility between water and a hydrophobic waterproof coating forming agent, does not occur any precipitation although an acid dye is added to an aqueous solution containing a hydrophobic polymer, and can exhibit good dyeing effect with forming a strong waterproof coating. Therefore, the semi-permanent hair dyeing composition of the present invention has an advantage in that since the dye fastened to the hair surface is not dissolved by rainwater or sweat, it can exhibit excellent hair protection effect without occurring contamination of skin and clothes. The present invention will now be described in detail with reference to the following examples, which are not intended to limit the scope of the present invention.

### EXAMPLE 1

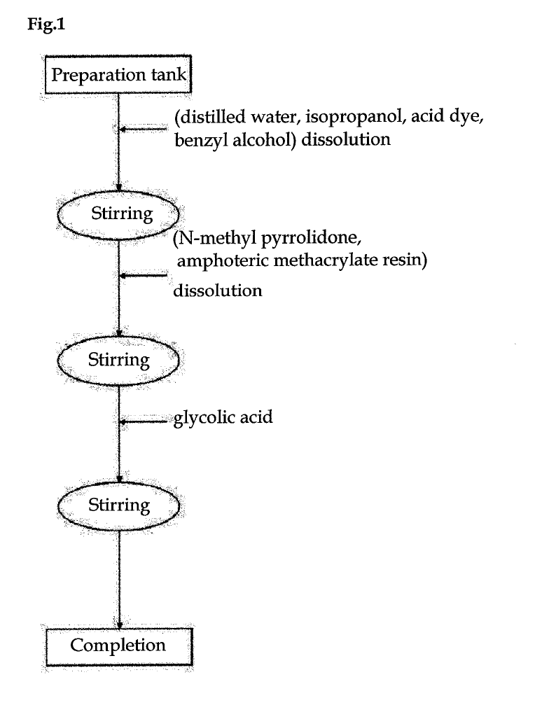

According to the procedure as shown in FIG. 1, as an acid dye, 0.4 wt % of violet 401 (Acid Violet 43, CAS No. 4430-18-6), 0.6 wt % of black 401 (Acid Black 1, CAS No. 1064-48-8), 0.5 wt % of orange 205 (Acid Orange 7, CAS No. 633-96-5), 0.4 wt % of yellow 406 (KI 406, CAS No. 587-98-4), 0.3 wt % of yellow 4 (Yellow 5, CAS No. 1934-21-0) and 0.1 wt % of red 227 (CI 17200, 3567-66-6) were dissolved in a mixture of 34.0 wt % of iso-propyl alcohol, 1.0 wt % of benzyl alcohol and 20.0 wt % of distilled water. To the resulting mixture were added 10.0 wt % of N-methyl pyrrolidone as a polymer solvent, 30.0 wt % of methacryloyl ethyl betaine/acrylate copolymer resin (trade name: Yukaformer 202, Mitsubishi Chemical Corporation) as a hydrophobic waterproof coating forming agent, and 3.0 wt % of glycolic acid as a pH regulator in order under stirring, to thereby prepare a hair dyeing composition in a liquid type.

### EXAMPLE 2

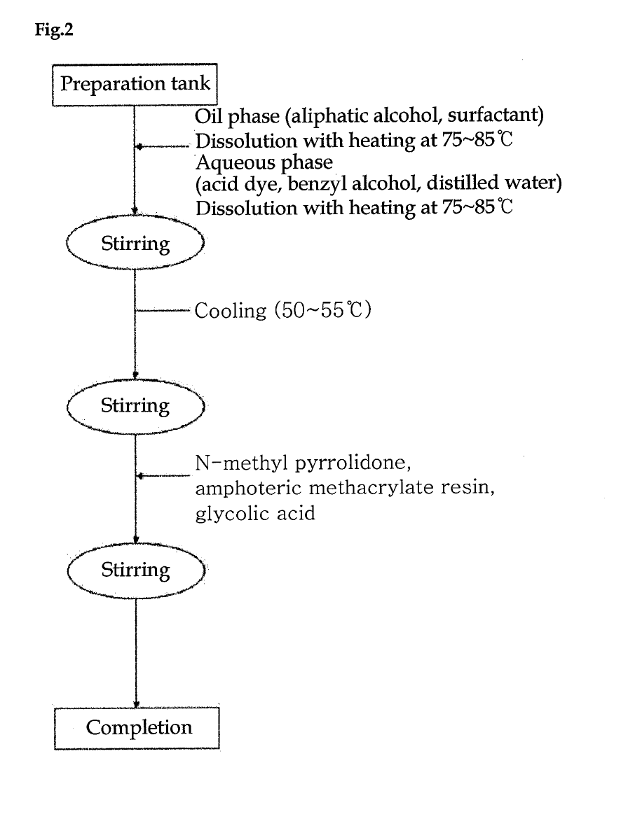

According to the procedure as shown in FIG. 2, 5.0 wt % of cetyl alcohol, 5.0 wt % of stearyl alcohol and 2.0 wt % of oleyl alcohol as an aliphatic alcohol, and 15.0 wt % of polyoxyethylene cetostearyl ether as a surfactant were dissolved under heating at a temperature of 75 to 85° C. As an acid dye, 0.4 wt % of violet 401 (Acid Violet 43, CAS No. 4430-18-6), 0.6 wt % of black 401 (Acid Black 1, CAS No. 1064-48-8), 0.5 wt % of orange 205 (Acid Orange 7, CAS No. 633-96-5), 0.4 wt % of yellow 406 (KI 406, CAS No. 587-98-4), 0.3 wt % of yellow 4 (Yellow 5, CAS No. 1934-21-0) and 0.1 wt % of red 227 (CI 17200, 3567-66-6) were dissolved in a mixture of 1.0 wt % of benzyl alcohol and 27.0 wt % of distilled water under heating at a temperature of 75 to 85° C. The resulting mixture was stirred for a while and cooled down to 50 to 55° C. To the resulting mixture were added 10 wt % of N-methyl pyrrolidone as a polymer solvent, 30.0 wt % of methacryloyl ethyl betaine/acrylate copolymer resin (trade name: Yukaformer 202, Mitsubishi Chemical Corporation) as a hydrophobic waterproof coating forming agent, and 3.0 wt % of glycolic acid as a pH regulator in order under stirring, to thereby prepare a hair dyeing composition in a cream type.

### TEST EXAMPLE

### Formation of a Waterproof Coating

In order to examine the ability of forming a waterproof coating of the hair dyeing compositions prepared in Examples 1 and 2, each composition was spread on the hair and a clean glass plate and dried at 20° C. for 10 minutes. After the hair and glass plate were left alone under the conditions of 60% relative humidity at 20° C. for 24 hours, they were soaked in clean water and an artificial sweat solution, respectively, and whether the dye is dissolved in water was examined. Here, the composition having no waterproof coating forming agent was used as a control. The artificial sweat solution was prepared by using 1.0 wt % of sodium chloride, 0.1 wt % of lactic acid, 0.1 wt % of disodium hydrogen phosphate, 0.02 wt % of histidine HCl and 98.78 wt % of distilled water.

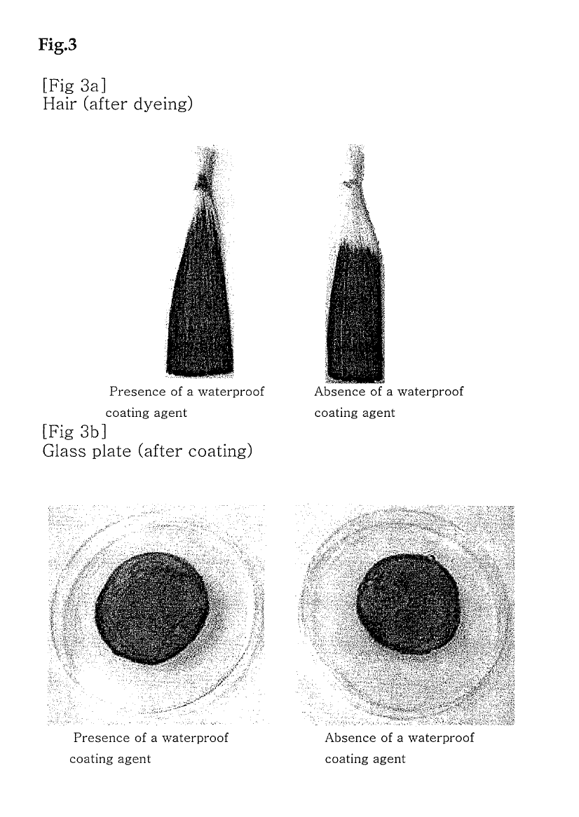

As can be seen in FIGS. 3a and 3b, the hair and glass plate left alone under the conditions of 60% relative humidity at 20° C. for 24 hours were the same regardless of the presence or absence of a waterproof coating forming agent.

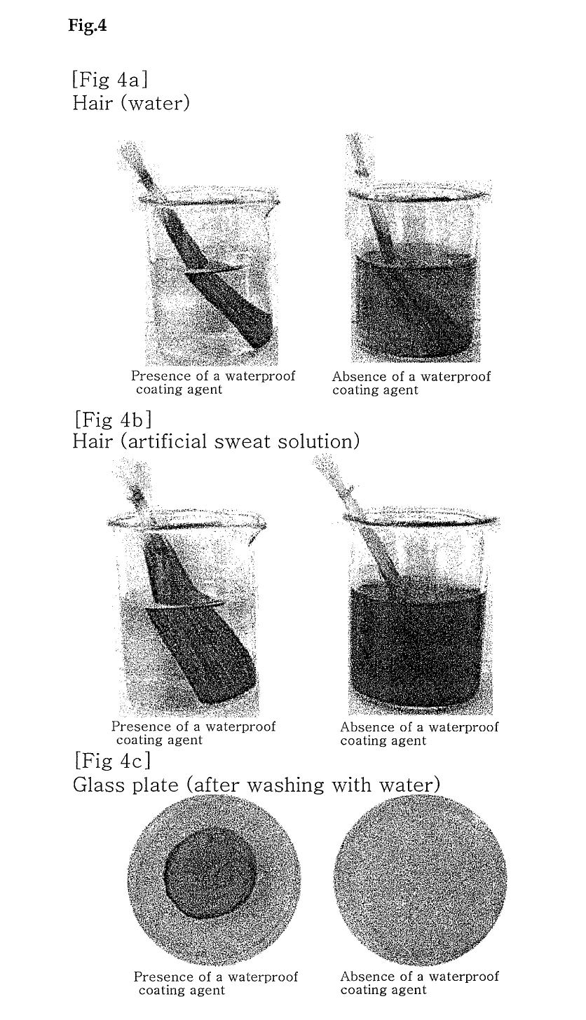

However, when each sample was soaked in water and the artificial sweat solution, as shown in FIGS. 4a, 4b and 4c, it has been found that while the dye was immediately dissolved out of the composition having no waterproof coating forming agent, the dye was not dissolved out of the composition of the present invention even after 1 hour of soaking.

The invention has been described in detail with reference to preferred embodiments thereof. However, it will be appreciated by those skilled in the art that changes may be made in these embodiments without departing from the principles and spirit of the invention, the scope of which is defined in the appended claims and their equivalents.

### INDUSTRIAL APPLICABILITY

As described above, since the semi-permanent hair dyeing composition using an acid dye according to the present invention comprises a hydrophobic waterproof coating forming agent and a unique polymer solvent, it can overcome the problem of compatibility between water and the hydrophobic waterproof coating forming agent, does not occur any precipitation although the acid dye is added to an aqueous solution containing a hydrophobic polymer, and can exhibit good dyeing effect with forming a strong waterproof coating. Therefore, the semi-permanent hair dyeing composition of the present invention has an advantage in that since the dye fastened to the hair surface is not dissolved by rainwater or sweat, it can exhibit excellent hair protection effect without skin and clothing contamination.

1. A semi-permanent hair dyeing composition using an acid dye comprising acrylate copolymer as a waterproof coating agent and N-methyl pyrrolidone as a solvent.

2. The semi-permanent hair dyeing composition according to claim 1, wherein the acrylate copolymer is methacryloyl ethyl betaine/acrylate copolymer.

3. The semi-permanent hair dyeing composition according to claim 1, which comprises 1.0-40.0 wt % of methacryloyl ethyl betaine/acrylate copolymer and 0.2-20.0 wt % of N-methyl pyrrolidone.
